(19) United States
(12) Patent Application Publication
(10) Pub. No.: US 2006/0078083 A1
(43) Pub. Date: Apr. 13, 2006

(54) X-ray dose compensation method and X-ray computed tomography apparatus

(75) Inventors: Akihiko Nishide, Tokyo (JP); Masatake Nukui, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/245,833

(22) Filed: Oct. 7, 2005

(30) Foreign Application Priority Data
Oct. 7, 2004 (JP) 2004-294707

(51) Int. Cl. H05G 1/60; A61B 6/00; G01N 23/00; G21K 1/12

(52) U.S. Cl. 378/16

(57) ABSTRACT

The present invention provides an X-ray dose compensation method for X-ray dose compensation of the detector signals from a multiple array detector with improved SNR, used for the detector signal detected by using the multiple array detector in which a plurality of X-ray detector channels placed in one dimensional array in the channel direction is stacked in a plurality of rows in the row direction to form X-ray detector channels of two dimensional matrix. A plurality of specific X-ray detector channels in the multiple array detector or X-ray planar detector or X-ray image intensifier are used as the X-ray dose reference channels for the X-ray dose compensation by means of signal sum or mean of detector signals from those X-ray dose reference channels.

### BACKGROUND OF THE INVENTION

The present invention relates to an X-ray dose compensation method and an X-ray CT (Computed Tomography) apparatus allowing compensating for X-ray dose of X-ray detection signal detected by using a multiple array detector or an X-ray planar detector or an X-ray image intensifier (I.I.), more specifically to an X-ray dose compensation method and an X-ray CT apparatus, which may allow improving the SNR (signal-to-noise ratio) of an X-ray tomographic image in all scan modes including the conventional scan and helical scan. The multiple array detector herein incorporates a plurality of lines of a plurality of X-ray detector channels arranged in one dimension in the direction of channel.

The X-ray CT apparatus using the multiple array detector has conventionally incorporated an X-ray dose detecting channel, i.e., X-ray dose reference channel in each row, so that the reference channel of each raw independently compensates for the X-ray dose of the main detector, i.e., the main channel (see for example the reference 1).

Reference 1: JP-A-2002-200071 (pp. 10-11, FIGS. 1-4)

In the X-ray CT apparatus using an multiple array detector or an X-ray planar detector or an X-ray image intensifier, when the slice thickness is thinner, then the slice thickness of X-ray dose reference channel also is thinner, and the X-ray dose of the detector signal from the main detector of thin slice needs to be compensated for by the detector signal of the X-ray dose reference signal having an inferior SNR, thus the SNR deteriorates more in the detector arrangement of thinner slice, and the noise increases more on the reconstructed image.

### SUMMARY OF THE INVENTION

Therefore, an object of the present invention is to provide a method for compensating for the X-ray dose with higher SNR of the detector signals from the multiple array detector, and an X-ray CT apparatus which compensates the X-ray dose using the same.

(1) In an aspect for solving the above problem, the present invention provides an X-ray dose compensation method, for compensating for the detection signal detected by using a multiple array detector or an X-ray planar detector or an X-ray image intensifier in which a plurality of X-ray detection channels arranged in the channel direction are arranged in a plurality of rows in the row direction and the X-ray detection channels are located in a form of matrix, the method comprises the step of: designating some of a plurality of X-ray detector channels in the multiple array detector or X-ray planar detector or X-ray image intensifier as the X-ray dose reference channels, andusing the signal derived based on the sum or mean of the detection signals from the X-ray dose reference channels to compensate for the X-ray dose.

(2) In another aspect for solving the above problem, the present invention provides: An X-ray CT apparatus, in which X-ray dose is compensated for the detection signal of a plurality of views detected by using a multiple array detector or an X-ray planar detector or an X-ray image intensifier, in which a plurality of X-ray detector channels arranged in the channel direction are arranged in a plurality of rows in the row direction and the X-ray detector channels are located in a form of matrix, the X-ray CT apparatus performing image reconstruction based on the signals after compensation, the apparatus comprising: a compensating means, by being a plurality of specific X-ray detection channels as the X-ray dose reference channels in the multiple array detector or X-ray planar detector or X-ray image intensifier, to compensate for the X-ray dose using the signal based on the sum or mean of the detection signals from those X-ray dose reference channels.

It is preferable that the above X-ray dose reference channels be located at the same channel in each row in the each X-ray detector array, for obtaining appropriately signals for X-ray dose compensation. It is also preferable that the sum or mean of the above detection signal is the sum or mean of signals except for the signals from those X-ray dose reference channels located at both ends of X-ray detector in the row direction, for further obtaining appropriately signals for compensation.

It is preferable that the X-ray dose reference channels in each row of X-ray detectors may have a plurality of channels in the channel direction of the X-ray detector for further improving the SNR of signals for X-ray dose compensation. It is also preferable that the X-ray dose reference channels in each row of X-ray detectors may be located at both ends or in proximity of both ends of the X-ray detector in the channel direction for improving the stability by decreasing the probability of blocking the X-ray dose compensating channel by an object to be detected. It is further preferable that the X-ray dose reference channel is used as the X-ray detecting channel for X-ray collimator control in order to improve the efficiency of usage of X-ray detector.

It is preferable that the X-ray dose reference channels may be divided into a plurality of groups along with the row direction of the X-ray detector to determine the sum or mean of the detector signals for each of the groups in order to use only the signal of groups not including the X-ray dose reference channels having incident X-ray blocked by an obstacle on the X-ray transmission path, for avoiding the influence of blockage of incident X-ray by the obstacle.

It is preferable that the sum or mean of the detector signals may be the sum or mean of signals except for those of X-ray dose reference channels having incident X-ray blocked by the obstacle on the X-ray transmission path, so as to avoid the influence of the subject blocking the X-ray.

It is preferable that the presence of the obstacle may be determined, based on the informative signals obtained from an X-ray generator, by detecting whether or not the incident X-ray in the X-ray dose reference channels is blocked, for the stability and high precision of detection of the blockage of incident X-ray into the X-ray dose reference channel.

It is preferable that the X-ray dose compensation may be based on the information obtained from the X-ray generator in case of blockage of incident X-ray into all of the X-ray dose reference channels, for the purpose of stable operation of the X-ray dose compensation in any circumstances, even when the precision is somewhat deteriorated.

It is preferable that the informative signal obtained from the X-ray generator is the tube current or tube voltage or both, since the informative signal is related to the X-ray dose.

According to the present invention, since specific X-ray detector channels of each row in the multiple array detector or X-ray planar detector or X-ray image intensifier are used for the X-ray dose reference channels, and the signal based on the sum or mean of detector signals of these X-ray dose reference channels are used for the X-ray dose compensation, thus a preprocessed projection data having better SNR can be obtained. The image reconstruction performed in accordance with such projection data may deliver a high quality tomographic image, which has an improved SNR.

The informative signal obtained from the X-ray generator such as the tube current and tube voltage may be used for the decision whether or not incident X-ray to the X-ray dose reference channels or for use as the X-ray dose reference signals in case in which incident X-ray is blocked for all of the X-ray dose reference channels, based on the informative signals obtained from the X-ray generator such as tube current and tube voltage.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic block diagram illustrating an X-ray CT apparatus in accordance with an example of the best mode carrying out the invention (first embodiment).

FIG. 2 is a schematic diagram illustrating a multiple array detector and X-ray dose reference channels.

FIG. 3 is a schematic diagram illustrating the revolving of X-ray tube and the multiple array detector.

FIG. 4 is a flow diagram illustrating the overview of operation of the X-ray CT apparatus.

FIG. 5 is a flow diagram illustrating details of preprocessing.

FIG. 6 is a schematic diagram illustrating the multiple array detector and the X-ray dose reference channels.

FIG. 7 is a schematic diagram illustrating grouping of X-ray dose reference channels.

FIG. 8 is a schematic diagram illustrating the detector data profile of the X-ray dose reference channels.

FIG. 9 is a schematic diagram illustrating the detector data profile of the X-ray dose reference channels.

FIG. 10 is a schematic diagram illustrating the detector data profile of the X-ray dose reference channels.

FIG. 11 is a schematic block diagram of X-ray dose compensation in accordance with second embodiment.

FIG. 12 is a flow diagram of the X-ray dose signal selection operation in accordance with second embodiment.

### DETAILED DESCRIPTION OF THE INVENTION

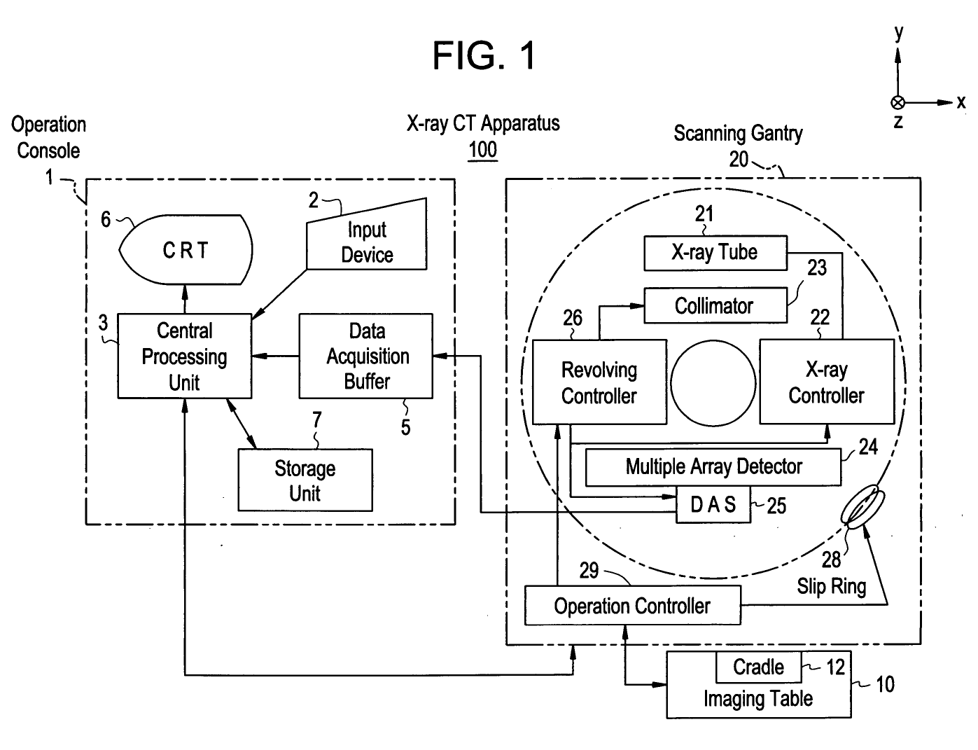

The best mode for carrying out the invention will be described herein below with reference to the drawings. It should be noted that the present invention is not to be considered to be limited to the best mode for carrying out the invention. FIG. 1 shows a block diagram of an X-ray CT apparatus. The apparatus is an exemplary embodiment for carrying out the invention. The arrangement of the present apparatus presents an example of the best mode for carrying out the invention in relation to the X-ray CT apparatus. The operation of the present apparatus presents an example of the best mode for carrying out the invention.

As shown in FIG. 1, the X-ray CT apparatus 100 incorporates an operation console 1, an imaging table 10, and a scanning gauntry 20. The operation console 1 includes an input device 2 for receiving input from the operator, a central processing unit 3 for executing such processes as image reconstruction, a data acquisition buffer 5 for collecting the projection data obtained from the scanning gantry 20, a CRT 6 for displaying a CT image reconstructed from the projection data, and a storage unit 7 for storing programs, data, and X-ray CT images.

The imaging table 10 includes a cradle 12 carrying a subject to be imaged thereon to move in and out of the bore (center void) of the scanning gantry 20. The cradle 12 may be translated vertically and laterally on the table by means of the motor built-in to the imaging table 10.

The scanning gantry 20 has an X-ray tube 21, an X-ray controller 22, a collimator 23, a multiple array detector 24, a DAS (data acquisition system) 25, a revolving controller 26 for revolving the X-ray tube 21 and the like around the body axis of the subject to be imaged, and an operation controller 29 for sending and receiving control signals to and from the operation console 1 and the imaging table 10.

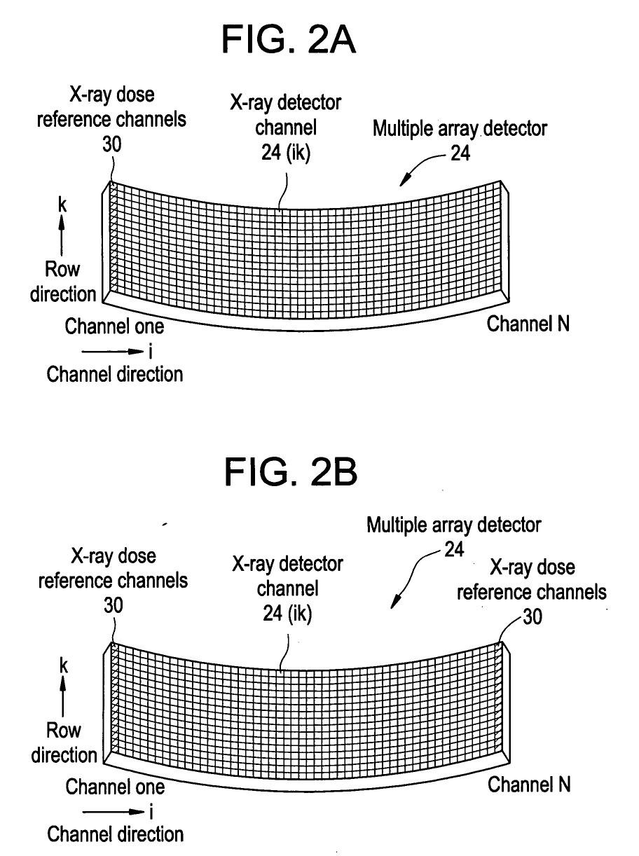

FIG. 2 shows a schematic arrangement of a multiple array detector 24. As shown in the figure, the multiple array detector 24 has a plurality of raw of a plurality of channels of X-ray detectors, having a plurality of X-ray detector channels 24 (ik) arranged in a two dimension matrix. The overall shape of the plurality of X-ray detector channels 24 (ik) forms an X-ray receptor plane concaved as an arc around the X-ray focal point.

Note that ‘i’ denotes the number of channels, e.g., i=1, 2, . . . , 1024. ‘k’ denotes the number of row, e.g., k=1, 2, . . . , 16. The X-ray detector channel 24 (ik) has detector rows, each having the same number of row k. The detector row of the multiple array detector 24 may not be limited to 16, but it can be any plural number. At or in the vicinity of either one end or both ends of the multiple array detector 24 the X-ray dose reference channels 30 are placed.

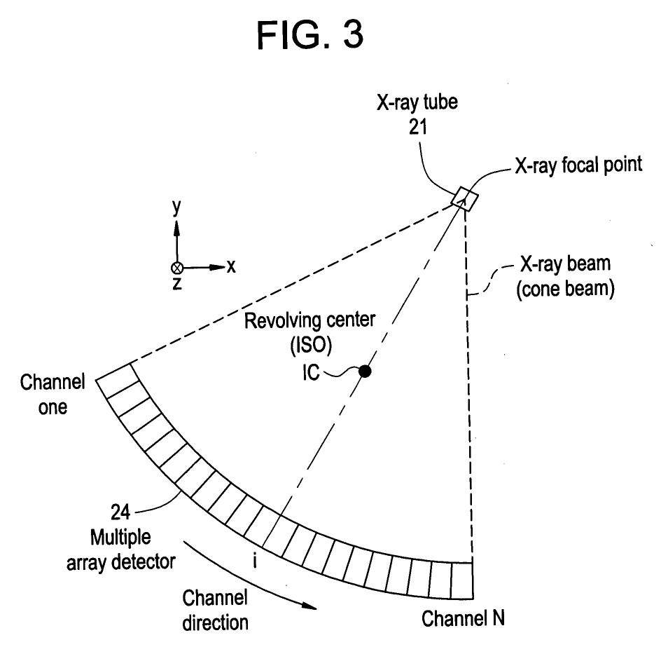

FIG. 3 describes the schematic diagram of the X-ray tube 21 and the multiple array detector 24. As shown in the figure, the X-ray tube 21 and the multiple array detector 24 revolves around the center pivot IC. When defining the vertical direction as y-axis, horizontal direction as x-axis, and the direction perpendicular to these directions as z-axis, the X-ray tube 21 and the multiple array detector 24 revolve in the x-y plane. The translational direction of the cradle 12 is z-axis. The X-ray tube 21 generates an X-ray beam from the X-ray focal point. The X-ray beam will be shaped to be a cone beam X-ray by the collimator 23 to irradiate the receptor plane of the multiple array detector 24.

### FIRST EMBODIMENT

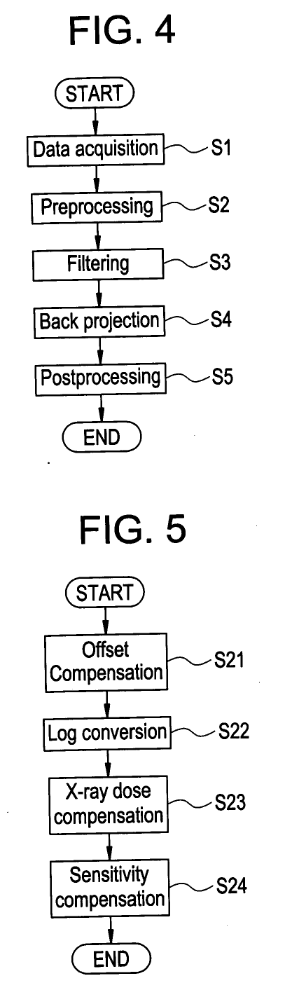

FIG. 4 shows a flow diagram indicating the overview of operation of the X-ray CT apparatus 100 in accordance with the first embodiment.

In step S1, when the X-ray tube 21 and the multiple array detector 24 are revolved around an object to be imaged while at the same time the cradle 12 is linearly translated, the projection data D0 (z, view, j, i) will be collected, where z is the linear translational position, view the view angle, j the number of detector row, and i the number of channel. Data acquisition in such a manner is performed for a helical scan. The data acquisition without the translational movement of the cradle 12 may be performed for a conventional scan (axial scan) or a cine-scan.

In step S2, a preprocess (offset correction, logarithm correction, X-ray dose correction, sensitivity correction) as shown in FIG. 5 is performed on the projection data D0 (z, view, j, i).

In step S3, the projection data D0 (z, view, j, i) preprocessed as above is filtered. More specifically, the data will be Fourier transformed, multiplied by a filter function (reconstruction function) in the frequency domain, and then invert Fourier transformed.

In step S4, the projection data D0 (z, view, j, i) thus filtered will be back projected to define a back projection data D3 (X, y).

In step S5, The back projection data D3 (X, y) will be postprocessed to convert to CT values to obtain a CT image.

FIG. 5 shows a flow diagram indicating the details of the preprocessing (step S2 of FIG. 4). In step S21, an offset correction is performed which decrements the offset data Doffset (ch, row) of each channel of the detector from the projection data D0. When defining the output signal as D21,
D21(ch, row)=D0(ch, row)−Doffset (ch, row).

In step S22, the X-ray projection data having offset corrected will be log-converted to obtain values proportional to the X-ray absorption index.

In step S23, data D22 having log-converted in step S22 is subtracted from the data Dref, which is indicative of the change in the X-ray output obtained in the X-ray dose reference channels 30, to obtain the output signal D23 of the step S23, i.e., the output signal having X-ray dose compensated.
D23(ch, row)=Dref−D22(ch, row)

In step S24, the output signal D24 of the step S24 will be given as follows, when using the sensitivity data Dsen of the multiple array detector 24, which data is previously determined:
D24(ch, row)=D23(ch, row)−Dsen(ch, row)

Here, when the data indicative of the change in the X-ray output is composed of Dref (ch, row), in the step 23, prior to the present invention, data of X-ray dose reference channels are averaged for N channels to obtain following equation.

$$\frac{1}{N} {\sum }_{i = 1}^{N} {D}_{Ref} ( ch , row ) \qquad EQ 1$$

Then the data value thus obtained is used for the X-ray dose compensation for all of the channels of the multiple array detector.

In the present apparatus, on the other hand, the data from X-ray dose reference channels for M rows are also to be averaged to obtain following equation.

$$\frac{1}{N \cdot M} {\sum }_{j = 1}^{M} {\sum }_{i = 1}^{N} {D}_{Ref} ( ch , row ) \qquad EQ 2$$

Moreover, the data value thus obtained is used for the X-ray dose compensation of all of the channels and all of the rows of the multiple array detector. In such a manner the SNR of the projection data after the X-ray dose compensation will be improved, allowing also improving the SNR of the tomographic image to be image reconstructed. The data of X-ray dose reference channels may also be simply added together instead of averaging. The improvement of SNR can be similarly achievable in such a way. The X-ray dose compensation in accordance with the step S23 may be performed on the central processing unit 3. The central processing unit 3 is an exemplary embodiment of the compensation means in accordance with the present invention.

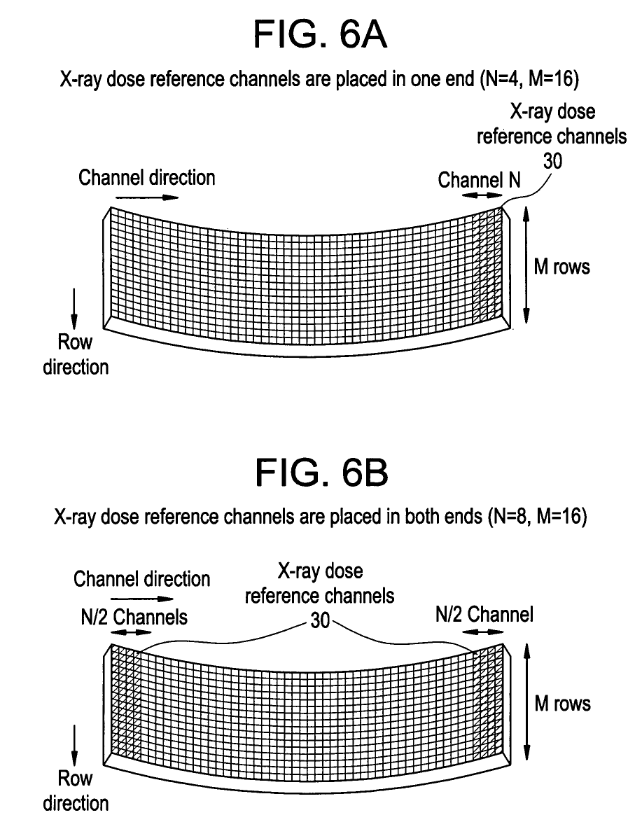

The X-ray dose reference channels 30, as shown in FIG. 6 (1) or (2), are located at one side or both sides of the multiple array detector 24, with N channels for each row. For example, in a multiple array X-ray CT of 16 rows, when compared with the prior art having reference channels made by averaging 4 channels of reference channels independently for each row, the present invention, which adds data for 16 rows by 4 channels to obtain Dref (ch, row), may improve 16 folds of the count values of the projection data, and 4 folds of SNR. The SNR is obviously improved as such.

The X-ray dose reference channels 30 are placed relatively at the same position in each detector array, so that the signal for compensation can be appropriately obtained. In addition, a plurality of the X-ray dose reference channels 30 are adjoined together in the channel direction of the X-ray detector in each detector array, the mean or addition of those signals may further improve the SNR of compensation signals. By placing the X-ray dose reference channels at the both ends in the channel direction of X-ray detector in each detector array, the probability that a compensation channel is blocked by the subject may be decreased to improve the stSince the X-ray in the z-axis is attenuated by the collimator control, the rows at both ends in the direction of z-axis (in the k direction) may have the possibility of lack of X-ray, resulting in some vulnerable error. The X-ray dose compensation can be done by Dref, which can be determined without the data from the rows at both ends.

It is alternatively possible that the collimator control may be performed by using these data from the rows of both ends. In addition, when performing the collimator control in this way, a higher precision of collimator control can be achieved by using the detector channels at both sides in the i direction of the multiple array detector 24. In this situation, the channels are also used for X-ray detector channels, yielding a higher efficiency of use of the detector.

It is quite possible that some of the X-ray dose reference channels 30 may have incident X-ray blocked by the body mass or position of the object to be imaged. This situation is also called as incident X-ray failure. Data from the X-ray detector channels having incident X-ray blocked is tend to be incorrect, causing the addition and average of all of the X-ray dose reference channels to be incorrect, thus preventing the proper X-ray dose compensation from achieving.

In order to address to such a situation, the central processing unit 3 monitors the presence of incident X-ray failure in the X-ray dose reference channels 30. When an incident X-ray failure occurs, it will perform X-ray dose compensation with proper data from the X-ray dose reference channels, by excluding the incorrect data of X-ray dose reference channels.

The detection of the presence of incident X-ray failure may be performed based on the tube current value, which is always monitored by the central processing unit 3, or based on the difference from the previous view data, or based on the difference from the data of X-ray dose reference channels in the next row or any other row, to determine the presence of data anomaly in individual X-ray dose reference channels 30 data.

Detector data that is determined to be incorrect will be excluded one by one from the computation and the sum or mean of the rest of the data will be used for the X-ray dose compensation. When using the sum, the summed value will be normalized in accordance with the percentage of excluded data in the entity.

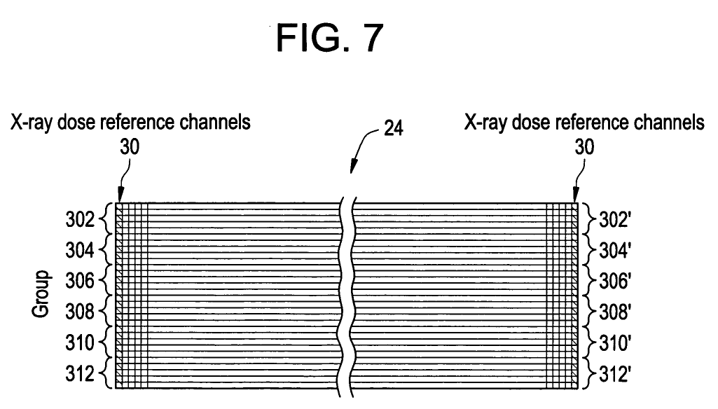

It is quite possible to perform the X-ray dose compensation by, instead of excluding detector data one by one from the computation, as shown in FIG. 7, dividing the X-ray dose reference channels 30 into a plurality of groups 302-312′ to determine, for each group, the sum or mean of detector signals to use the signal in the groups that do not include the X-ray dose reference channels having incident X-ray blocked.

The detection of incident X-ray failure and the exclusion of improper data may be performed based on the profile of detector data in the row direction (k direction) of the X-ray dose reference channels 30. This will be described in the following.

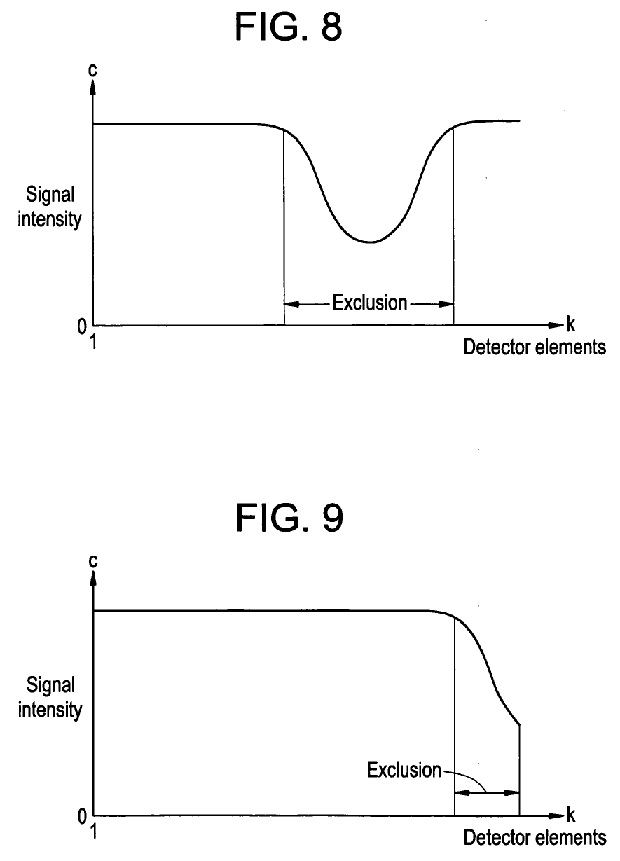

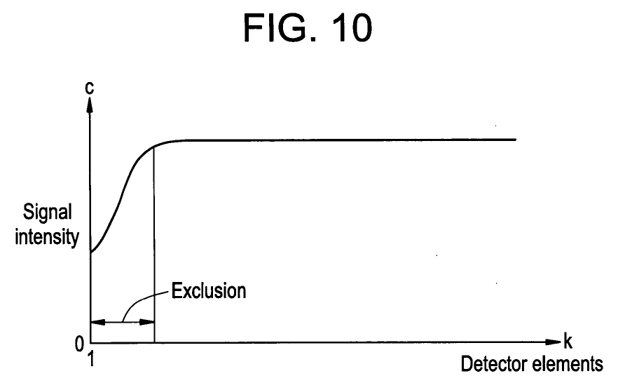

In the case of incident X-ray failure, the data profile of the X-ray dose reference channels 30 in the row direction can be for example as shown in FIG. 8. More specifically, in the data profile, there will be a dip of signal intensity caused by the X-ray incident failure. The dip of signal intensity may be formed as shown in FIG. 9 or FIG. 10, depending on the position of incident X-ray failure.

The dip of signal intensity caused by the incident X-ray failure corresponds to the projection of the object being imaged on the X-ray dose reference channels 30. The signal has a specific pattern of continuous deficiency of signal intensity in the dip. This characteristics may be used for specifying the range of incident X-ray failure in the X-ray dose reference channels 30. The detector data belonging to this area can be excluded from the computation of the sum or mean.

### SECOND EMBODIMENT

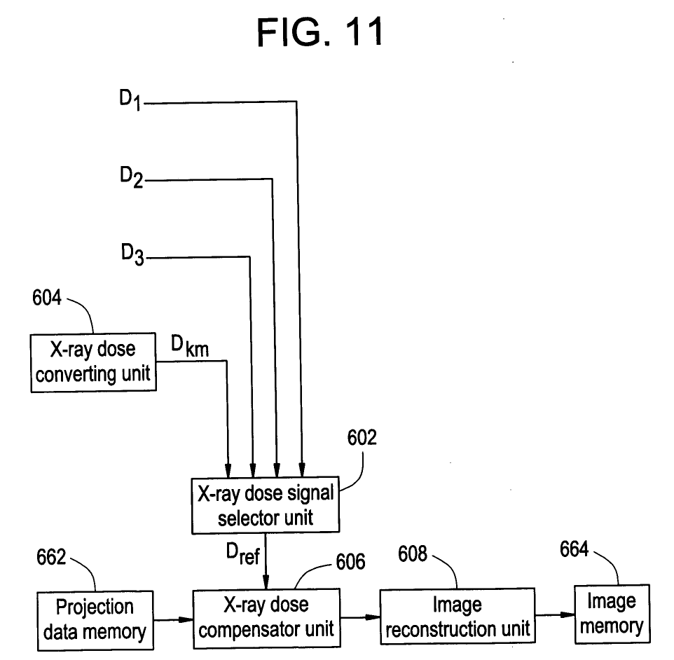

FIG. 11 shows a schematic block diagram of the part of apparatus involving the X-ray dose compensation. As shown in FIG. 11, the apparatus comprises an X-ray dose signal selector unit 602. The X-ray dose signal selector unit 602 may be achieved by the capability of central processing unit 3.

To the X-ray dose signal selector unit 602, input are series of data D1, D2, D3 and Dkm, indicative of X-ray dose. Data D1 is the data indicative of X-ray dose detected by the left hand channels in the X-ray dose reference channels 30. Data D2 is the data indicative of X-ray dose detected by the right hand channels in the X-ray dose reference channels 30. Data D3 is the data indicative of X-ray dose detected by the X-ray detector channels in the left or right hand vicinity of X-ray dose reference channels 30. Data Dkm is the data indicative of X-ray dose, converted by an X-ray dose converting unit 604, of the information of the X-ray tube current or tube voltage obtained from the X-ray controller 22 of the X-ray generator.

The X-ray dose converting unit 604 converts the X-ray dose based on the tube voltage signal or tube current signal collected from the X-ray controller 22, part of the X-ray generator, by the data acquisition system DAS 25. The X-ray dose converting unit 604 can be achieved by the capability of the central processing unit 3. The X-ray dose converting unit 604 is an exemplary embodiment of the converter means in accordance with the present invention. The conversion of X-ray dose, or data transformation may be done by using a data table and the like, which stores the relationship between the X-ray dose and the combinations of tube voltage and tube current. The relationship between the combinations of tube voltage and tube current and the X-ray dose can be predetermined by practically measuring the X-ray dose with combinations of tube voltage and tube current used for the imaging, so that the same unique data table is used every time. Alternatively, the data table can be updated by the calibration at any given time to achieve the conversion or data transformation of much higher precision.

Data items D1 and D2 are data indicative of X-ray dose detected by the X-ray dose reference channels 30, respectively, which data items represent the X-ray dose incident to the multiple array detector 24 with the high fidelity.

Data D3 is the data indicative of X-ray dose in the vicinity of the X-ray dose reference channels 30, detected by the multiple array detector 24, which data item represents the X-ray dose with the fidelity as high as data items D1 and D2. Data item Dkm is the data indicative of X-ray dose converted by the X-ray dose converting unit 604, which data is not actually measured, however can be used, by equally considering similar to other data items of X-ray dose.

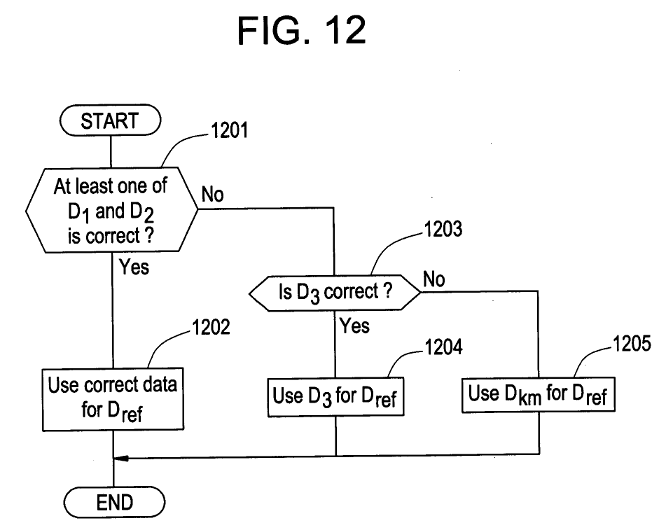

FIG. 12 shows a flow diagram of X-ray dose signal selection operation performed by the X-ray dose signal selector unit 602. As shown in FIG. 12, in step 1201, at least one of data items D1 and D2 is determined whether or not to be correct. The determination whether or not the data is correct may be performed as follows, based on an appropriate threshold predetermined.

More specifically, when defining

Dref (n): X-ray dose reference data for n views, or D1 or D2

Dkm (n): X-ray dose conversion data based on the information from the X-ray generator for n views, or Dkm,

then, if
1−ε<Dref(n)/Dkm(n)<1+ε,

the data will be good if the Dref (n)/Dkm (n) falls within the threshold ε of error, and will be abnormal if out of threshold, resulted from such a cause as the incident X-ray is blocked by an obstacle on the X-ray transmission path.

Similarly, when the multiple array detector 24 has a plurality of X-ray detector arrays, each of data D1 and D2 will have a plurality of channels, the correctness of data will be then determined for the mean of channels for each channel or for each subgroup of channels.

If there is at least one correct channel or one correct subgroup of channels, then in step 1202, that data will be marked as the data for X-ray dose compensation, or X-ray dose reference data Dref. In such a manner, the X-ray dose reference data of the highest precision can be stably obtained.

If there is not a correct data item within data D1 and D2, then in step 1203 the data D3 is determined whether or not to be correct. If data D3 is correct then in step 1204 the data will be used for the X-ray dose reference data Dref. In such a manner, an alternative data item of X-ray dose reference can be obtained even when every data D1 and D2 becomes unusable resulted from an obstacle that blocks the X-ray dose reference channels 30.

In case in which there is not a correct data in data D1 and D2, as well as no correct data in data D3, then in step 1205, data Dkm is used for the X-ray dose reference data Dref. In such a manner, at least the minimum alternative data usable of X-ray dose reference can still be obtained even when every data D1, D2 and D3 becomes unusable resulted from an obstacle that blocks the X-ray dose reference channel 30 and the multiple array detector 24.

As stated above, the priority selection of data D1, D2 and D3 as well as Dkm allows the most appropriate X-ray dose reference data to be obtained in accordance with the circumstances to establish a reasonable selection of X-ray dose reference data.

The X-ray dose reference data Dref thus selected is input into an X-ray dose compensator unit 606. The X-ray dose compensator unit 606 uses the X-ray dose reference data Dref to perform the X-ray dose compensation of the projection data read out from a projection data memory 662. The projection data memory 662 corresponds to part of a storage device 66.

The most appropriate X-ray dose reference data Dref is selected in accordance with the circumstances, to positively perform the X-ray dose compensation. The X-ray dose compensator unit 606 may be achieved by the capability of the central processing unit 3. If there is a plurality of channels for the data D1 and D2 selected as the X-ray dose reference data Dref, then the mean value of the data is used for the X-ray dose compensation. The part composed of the X-ray dose signal selector unit 602 and the X-ray dose compensator unit 606 is an exemplary embodiment of the best mode carrying out the compensation means in accordance with the present invention.

The X-ray dose compensation may be performed based on the equation (1) or (2) below. The equation (1) is for the case in which every data has been log-converted, while the equation (2) is for the case in which no data has been log-converted.

EQ 3
Dc(i)=Dref−Dm(i); after log conversion (1)

EQ 4
Dc(i)=Dref/Dm(i); prior to log conversion (2)
where
Dc (i): data after X-ray dose compensation;
Dm (i) data prior to X-ray dose compensation; and
Dref: X-ray dose reference data.

The projection data that has been compensated for is used for image reconstruction. The image reconstruction uses the compensated projection data to reconstruct an image by the filter compensation back projection method and the like. The image reconstruction may be achieved by the capability of the central processing unit 3. The image thus reconstructed may be stored in an image memory 664. The image memory 664 corresponds to part of the storage unit 7.

In the arrangement shown in FIG. 11, either input for data D1 or D2 may be omitted. Also in the arrangement shown in FIG. 11, the input for data D3 may be omitted. Furthermore, in the arrangement shown in FIG. 11 the input for data Dkm may be omitted. In summary, the system needs at least two data sources having different types from each other of X-ray dose compensation.

In accordance with the X-ray CT apparatus 100 as have been described above, The X-ray dose compensation with improved SNR may obtain the preprocessed projection data with improved SNR. The image reconstruction based on such projection data may reconstruct a higher quality tomographic image with improved SNR.

As stated above, in an X-ray CT which does not perform a hardware-based fan-parallel conversion but does collect fan data, since data is collected for all rows of all channels in the multiple array detector simultaneously, only one X-ray dose reference channel is necessary for all rows of all channels. This has not been applied to the X-ray dose compensation.

Although the preferred embodiments use the multiple array detector, the X-ray dose compensation similar thereto may be achievable in an X-ray CT apparatus incorporating an X-ray planar detector such as a flat panel, or an X-ray image intensifier.

Many widely different embodiments of the invention may be configured without departing from the spirit and the scope of the present invention. It should be understood that the present invention is not limited to the specific embodiments described in the specification, except as defined in the appended claims.

1. An X-ray dose compensation method, for compensating for the detection signal detected by using a multiple array detector or an X-ray planar detector or an X-ray image intensifier in which a plurality of X-ray detection channels arranged in the channel direction is arranged in a plurality of rows in the row direction and the X-ray detection channels are located in a form of matrix, said method comprises the step of:
designating some of a plurality of X-ray detector channels in said multiple array detector or X-ray planar detector or X-ray image intensifier as the X-ray dose reference channels, and
using the signal derived based on the sum or mean of the detection signals from said X-ray dose reference channels to compensate for the X-ray dose.

2. An X-ray dose compensation method according to claim 1, wherein
said X-ray dose reference channels are located in the same channel position in each row in each X-ray detector array.

3. An X-ray dose compensation method according to claim 1, wherein:
the sum or mean of said X-ray dose reference signals is the sum or mean of said X-ray detection signals except for those derived from the X-ray dose reference channels of both ends in the row direction of detectors.

4. An X-ray dose compensation method according to claim 1, wherein:
said X-ray dose reference channels have a plurality of channels in the channel direction of X-ray detectors in each X-ray detector array.

5. An X-ray dose compensation method according to claim 1, wherein:
said X-ray dose reference channels are located to both ends or in proximity to both ends of each X-ray detector array in the channel direction of X-ray detectors.

6. An X-ray dose compensation method according to claim 2, wherein:
said X-ray dose reference channels are also used as the X-ray detector channels for X-ray collimator control.

7. An X-ray dose compensation method according to claim 1, comprising the steps of:
dividing said X-ray dose reference channel into a plurality of groups in the direction of each X-ray detector array to determine the sum or mean of said detector signals for each group; and
using the signal of groups not including the X-ray dose reference channel having incident X-ray blocked by an obstacle on the X-ray transmission path.

8. An X-ray dose compensation method according to claim 1, wherein:
the sum or mean of said X-ray dose reference signals is the sum or mean of signals except for those derived from the X-ray dose reference channels having incident X-ray blocked by an obstacle on the X-ray transmission path.

9. An X-ray dose compensation method according to claim 7, wherein:
the presence of said obstacle is determined, based on informative signals obtained from an X-ray generator, by detecting whether or not the incident X-ray in the X-ray dose reference channel is blocked.

10. An X-ray CT apparatus, wherein X-ray dose is compensated for the detection signal of a plurality of views detected by using a multiple array detector or an X-ray planar detector or an X-ray image intensifier, in which a plurality of X-ray detection channels arranged in the channel direction is arranged in a plurality of rows in the row direction and the X-ray detection channels are located in a form of matrix, said X-ray CT apparatus performing image reconstruction based on the signals after compensation, said apparatus comprising:
a compensating device by being a plurality of specific X-ray detection channels as the X-ray dose reference channels in said multiple array detector or X-ray planar detector or X-ray image intensifier, to compensate for the X-ray dose using the signal based on the sum or mean of the detection signals from those X-ray dose reference channels.

11. An X-ray CT apparatus according to claim 10, wherein:
said X-ray dose reference channels are located in the same channel position in each row in each X-ray detector array.

12. An X-ray CT apparatus according to claim 10, wherein:
the sum or mean of said X-ray dose reference signals is the sum or mean of said X-ray detection signals except for those derived from the X-ray dose reference channels of both ends in the row direction of detectors.

13. An X-ray CT apparatus according to claim 10,
said X-ray dose reference channels have a plurality of channels in the channel direction of X-ray detectors in each X-ray detector array.

14. An X-ray CT apparatus according to claim 10, wherein:
said X-ray dose reference channels are located to both ends or in proximity to both ends of each X-ray detector array in the channel direction of X-ray detectors.

15. An X-ray CT apparatus according to claim 11, wherein:
said X-ray dose reference channels are also used as the X-ray detector channels for X-ray collimator control.

16. An X-ray CT apparatus according to claim 10, wherein:
said compensating device comprises the steps of:
dividing said X-ray dose reference channel into a plurality of groups in the direction of each X-ray detector array to determine the sum or mean of said detector signals for each group; and
using the signal of groups not including the X-ray dose reference channel having incident X-ray blocked by an obstacle on the X-ray transmission path.

17. An X-ray CT apparatus according to claim 10, wherein:
the sum or mean of said X-ray dose reference signals is the sum or mean of signals except for those derived from the X-ray dose reference channels having incident X-ray blocked by an obstacle on the X-ray transmission path.

18. An X-ray CT apparatus according to claim 16, wherein:
the presence of said obstacle is determined, based on informative signals obtained from an X-ray generator, by detecting whether or not the incident X-ray in the X-ray dose reference channel is blocked.

19. An X-ray CT apparatus according to claim 18, wherein:
said X-ray dose compensation is performed based on the informative signal obtained from the X-ray generator in case in which the incident X-ray for all X-ray dose reference channels is blocked.

20. An X-ray CT apparatus according to claim 19, wherein:
information derived from the X-ray generator about the presence of said obstacle is the tube current or tube voltage or both.
